(19) United States
(12) Patent Application Publication
(10) Pub. No.: US 2012/0109054 A1
(43) Pub. Date: May 3, 2012

(54) DEVICES WITH AN ERODIBLE SURFACE FOR DELIVERING AT LEAST ONE ACTIVE AGENT TO TISSUE OVER A PROLONGED PERIOD OF TIME

(75) Inventors: Robert F. Thompson, Weston, MA (US); Charles D. Leahy, Concord, MA (US); Edward J. Ellis, Lynnfield, MA (US)

(73) Assignee: VISTA SCIENTIFIC LLC, Andover, MA (US)

(21) Appl. No.: 13/284,897

(22) Filed: Oct. 29, 2011

Related U.S. Application Data
(60) Provisional application No. 61/408,022, filed on Oct. 29, 2010.

(51) Int. Cl. A61M 5/00

(52) U.S. Cl. 604/93.01

(57) ABSTRACT

A device is disclosed for delivering an active agent to target tissue at a site that includes a bodily fluid. The device includes a body that has an erodible member that releases the active agent over a prescribed period of time.

### CROSS-REFERENCE TO RELATED APPLICATIONS

The present application claims the benefit of U.S. Patent application serial No. 61/408,022, filed Oct. 29, 2010, each of which is hereby incorporated by reference in its entirety.

### STATEMENT REGARDING FEDERAL SPONSORSHIP

The U.S. Government has a paid-up license in this invention and the right in limited circumstances to require the patent owner to license others on reasonable terms as provided for by the terms of grant #2 R44 EY013479-04 awarded by the National Institutes of Health.

### TECHNICAL FIELD

The present invention also generally pertains to devices for prolonged delivery of active agents (e.g., pharmaceuticals) to body tissue. Additionally, it pertains to controlling the release of the active agent from a device. More particularly, but not by way of limitation, the present invention pertains to biocompatible devices for localized delivery of active agents to the eye.

### BACKGROUND

There are many different types of delivery devices that are used to deliver active agents, such as drugs, to a patient, including but not limited to capsules, implants, etc. One subclass of delivery devices is ocular delivery devices for delivering an active agent to the eye.

With respect to ocular drug delivery devices, approximately 90% of all ophthalmic drug formulations are applied as eye drops. In addition to being difficult for patients to insert accurately, the use of eye drops suffers from two major technical disadvantages, their rapid elimination from the eye and their poor bioavailability to the target tissues. As a result of tear film dilution and elimination and the permeability barriers of the cornea, typically significantly less than five percent of the applied dose of drug reaches the intraocular tissues. Topical ophthalmic pharmaceutical solutions are therefore formulated in high concentrations and require frequent dosing. Non-compliance with treatment, due to required frequency of dosing, lack of detectable symptom relief in immediate association with treatment application, undesirable systemic side effects due to the need for high concentrations of drug and other reasons, is a major clinical disadvantage.

To address these issues the idea of placing a solid device into or near the eye to deliver a beneficial agent for extended periods of time has attracted development work for many years. In general these devices can be characterized as matrix or depot type devices. The matrix device is composed of one material and the beneficial agent is contained throughout this material. A depot device contains the agent in one or more distinct portions of the device. These devices contain a depot of beneficial agent or a depot of material containing the beneficial agent also referred to as a drug depot, drug core, medication depot or simply, a depot. The space in the device's body that contains the depot is referred to by a variety of terms including well, pocket, cache, cavity, reservoir and chamber. U.S. Pat. No. 3,302,646 to Behney discloses a device for bovine ocular drug delivery. The device has a pocket filled with ointment that is held adjacent to the corneal surface and front scleral surface of the eye.

More typically the depot is internal to the device and much of the prior art in these kinds of devices is focused on transporting the drug from the depot to the surface of the device or managing the rate of transport to the dispensing surface.

U.S. Pat. No. 3,416,530 to Ness discloses the use of perforations with capillary action to bring drug to the device's surface from its internal reservoir. U.S. Pat. No. 4,186,184 to Zaffaroni discloses a device with a delivery portal open to that surface of the device that is deemed to be most appropriate for the tissue being targeted.

U.S. Pat. No. 4,973,304 to Graham, et al discloses the use of hydrogel ports to transport drug from the reservoir to the surface of the device.

U.S. Pat. No. 5,902,598 to Chen, et al discloses a device with a diffusion port to transport drug from the reservoir to the surface of the device.

For many drugs and delivery systems, only a small pocket with drug or a larger pocket with a small exterior dispensing surface is all that is necessary to provide therapeutic levels of drug for extended periods. Drug is released from these systems via a distinct opening or portal on the device that is immediately adjacent to ocular tissue. However, concentrating the drug release into a narrow portion of ocular tissue is not usually therapeutically optimal and is of some concern, particularly with drugs with known side effects such as inflammation, and especially in the cases where the device is relatively immobile in relation to the immediately adjacent tissue.

Therefore, a need exists in the ocular drug delivery field for a drug delivery device capable of incorporating a drug depot and from that depot, broadening the drug release over a greater portion of ocular tissue. Ideally, such a device should be capable of delivering a wide variety of agents to treat or benefit physical conditions and should be relatively easy to manufacture. In addition the relatively smooth surfaces of matrix devices would benefit from tear flow features that increased and improved the devices surface area and increased drug acquisition and dispersal.

### SUMMARY

In one embodiment, the present invention generally pertains to devices for prolonged delivery of active agents (e.g., pharmaceuticals) to body tissue (target tissue). Additionally it pertains to controlling the release of the active agent from the delivery device. More particularly, but not by way of limitation, the present invention pertains to biocompatible devices for localized delivery of pharmaceuticals to the eye. In this ocular application, the invention would be useful in the configuration of ocular inserts, punctal plugs, ophthalmic implants, contact lenses and other ocular devices configured, at least in part, for drug delivery.

The present invention refers to controlling medication release from either a matrix type or a depot type, drug delivery device. There are two operative approaches to the practice of this invention; one applies to a matrix type device, that is, a device that is constructed entirely of a medication containing matrix. The second approach involves a depot type device, where the medication is localized within the device body. Additionally, a device could be a combination of matrix type and depot type. One application of such a device would be to deliver different drugs simultaneously.

The devices of the present invention better manage the release kinetics and drug delivery throughout the device's therapeutic residence. The device can be configured to gradually expose more drug dispensing surface area over time. As the device begins to dispense drug, a limited amount of unrestricted surface area would be exposed to bodily fluids and tissues and as a result a more limited amount of drug would be released initially. Through the use of a bio-erodible covering of varying depth or thickness over potential dispensing surfaces, erosion would gradually cause more surface area to be exposed and thereby maintain a more gradual decrease in release rate than would occur from a dispensing surface of fixed dimension.

In one aspect, the present invention includes a drug delivery device including a structural body whereby medication is present throughout the body or localized within the body and the medication is released to the environment outside of the body through one or more surfaces of the body.

### BRIEF DESCRIPTION OF THE DRAWING FIGURES

FIG. 1A is a top view of one exemplary device for delivering an active agent to target tissue over a prolonged period of time;

FIG. 1B is a side view of the device of FIG. 1A;

FIG. 2A is a top view of a single modified drug depot for delivering an active agent to target tissue over a prolonged period of time, the depot being supported by the delivery device;

FIG. 2B is a cross sectional side view of the modified drug depot of FIG. 2A;

FIG. 3 is a perspective view of one exemplary device for delivering an active agent to target tissue over a prolonged period of time; and

FIG. 4 is a representation of one exemplary device for delivering an active agent to target tissue.

### DETAILED DESCRIPTION OF CERTAIN EMBODIMENTS

The present application discloses a number of devices for delivering an active agent, which can be in the form of a drug(s) and/or a therapeutic agent, and/or other beneficial agent, to target tissue. The devices are intended for placement in a patient proximate target tissue to which the active agent is delivered. It will be appreciated that the devices can be placed in any number of different locations within the body and therefore, the target tissue can be different tissue found throughout the patient's body.

More specifically, the device 100, as well as the other devices disclosed herein and shown in the various figures, is constructed to deliver an active agent to target tissue. The expression “agent” as used herein broadly includes any compound, composition of matter, or mixture thereof that can be delivered from the device to produce a beneficial and useful result. For the purposes of this invention the term medication, medicinal agent, therapeutic agent, beneficial agent or drug can be taken as synonymous.

The devices described in this invention contain an active agent effective in obtaining a desired local or systemic physiological or pharmacological effect. The following classes of active agents can be incorporated into the devices of the present invention.

Suitable drugs or active agents that can be utilized with the present delivery devices include, by way of example only, but are not limited to: (A) Anti-infectives: such as antibiotics, including tetracycline, chlortetracycline, bacitracin, neomycin, polymyxin B, gramicidin, oxytetracycline, chloramphenicol, and erythromycin; sulfonamides, including sulfacetamide, sulfamethizole, sulfisoxazole; quinolones, including ofloxacin, norfloxacin, ciprofloxacin, sporfloxacin; aminoglycosides, including amikacin, tobramycin, gentamicin; cephalosporins; combinations of antibiotics; antivirals, including idoxuridine, trifluridine, vidarabine cidofovir, foscarnet sodium, ganciclovir sodium and acyclovir; antifungals such as amphotericin B, nystatin, flucytosine, fluconazole, natamycin, miconazole and ketoconazole; and other anti-infectives including nitrofurazone and sodium propionate; (B) Antiallergenics: such as antzoline, methapyriline, chlorpheniramine, pyrilamine and prophenpyridamine, emedastine, ketorolac, levocabastin, lodoxamide, loteprednol, naphazoline/antazoline, naphazoline/pheniramine, olopatadine and cromolyn sodium; (C) Anti-inflammatories: such as hydrocortisone, hydrocortisone acetate, dexamethasone, dexamethasone 21-phosphate, fluocinolone, medrysone, prednisolone, prednisolone 21-phosphate, prednisolone acetate, fluorometholone, fluorometholone acetate, meddrysone, loteprednol etabonate, rimexolone; (D) Nonsteroidal anti-inflammatories: such as flurbiprofen, suprofen, diclofenac, indomethacin, ketoprofen, and ketorolac; (E) Decongestants: such as phenylephrine, naphazoline, oxymetazoline, and tetrahydrazoline; (F) Miotics and anticholinesterases: such as pilocarpine, eserine talicylate, carbachol, diisopropyl fluorophosphate, phospholine iodide, and demecarium bromide; and (G) Mydriatics: such as atropine sulfate, cyclopentolate; homatropine, scopolamine, tropicamide, eucatropine, and hydroxyamphetamine.

Furthermore, the following active agents are also useful in the present devices: (A) Antiglaucoma agents: such as adrenergics, including epinephrine and dipivefrin, epinephryl borate; β-adrenergic blocking agents, including levobunolol, betaxolol, metipranolol, timolol, carteolol; α-adrenergic agonists, including apraclonidine, clonidine, brimonidine; parasympathomimetics, including pilocarpine, carbachol; cholinesterase inhibitors, including isoflurophate, demecarium bromide, echothiephate iodide; carbonic anhydrase inhibitors, including dichlorophenamide acetazolamide, methazolamide, dorzolamide, brinzolamide, dichlorphenamide; prostaglandins, including latanoprost, travatan, bimatoprost; diconosoids and combinations of the above, such as a β-adrenergic blocking agent with a carbonic anhydrase inhibitor; and (B) Anticataract drugs: such as aldose reductase inhibitors including tolerestat, statol, sorbinil; antioxidants, including ascorbic acid, vitamin E; nutritional supplements, including glutathione and zinc.

Yet another group of active agents is in the form of lubricants: such as glycerin, propylene glycol, polyglycerins and select water soluble polymers, such as the cellulosics, polyethylene oxides, polyethylene glycols and biopolymers such as hyaluronic acid and chitosan.

In addition to the above agents, other agents suitable for treating, benefitting, managing, or diagnosing ocular conditions may be utilized and administered using the sustained release drug delivery devices of the current invention.

In accordance with one aspect of the present invention, a device for dispensing active agent can consist of a body forming a carrier (body) and drug containing space (drug depot) with one or more dispensing surfaces incorporated onto or into specific surfaces of the device. The drug depot can consist of a three-dimensional space in or on the device containing one or more drugs or drug containing media. The devices of the present invention can be utilized for controlled ophthalmic drug delivery of substances to be distributed into the tear film for greater dispersal to the ocular tissues. Its design technology takes advantage of the physical forces created by blinking and eye movement and facilitates continuous exchange of tear fluid proximate the drug depot's dispensing surface. Its design can also include physical features that reduce or eliminate direct contact between the dispensing surfaces and adjacent tissue. The device configuration is useful for release of drugs such as a prostaglandin analog that might otherwise cause localized irritation, hyperemia, or hyperpigmentation. The device is also useful for a number of conditions including glaucoma, dry eye, infection, ocular surface disorders, and post-surgical healing. The device is particularly useful for releasing glaucoma medications directly into the tear film, thereby supplying drug both via the trans-corneal route into the anterior segment, and via a trans-conjunctival route with broad circumlimbal distribution outside the globe, proximal but external to the root of the iris, for penetration around the entire globe, to the targeted ciliary body and/or episcleral region surrounding the trabecular meshwork. Glaucoma medications are more effective when distributed efficiently to the entire anterior segment tissues that are the target of treatment.

Such delivery of drug is in contrast to concentrating the drug release directly against the tissue from a device with an opening of its drug depot directly over localized areas of tissue as is done with conventional drug delivery devices. Many dry eye medications that act on the ocular surface would also be more effective when distributed to large areas of the ocular surface through the flowing, dynamic tear film. Thus more even distribution to the entire ocular surface where the active agent is needed improves the treatment effect of a given amount of active agent released, while lessening potential toxicity to the tissue immediately adjacent to the opening of the drug depot. The invention when used in an ocular environment works towards more consistent drug release rates, using the tear film acting as an endless sink and active agent dispersion medium, by mixing the tear fluid over the device's drug depot and drawing the drug out of the drug depot, and presenting it via the tear film to large areas of target tissue. This tear fluid route of delivery relies on a concentration gradient between the tear film and the target tissue to help drive the active agent towards the target tissue, rather than on a strictly localized concentration gradient limiting delivery from the drug depot to that localized between the topical device's drug depot surface and the immediately proximal portion of the target tissue.

This delivery alternative can reduce the undesirable side effects of hyperemia, inflammation and hyperpigmentation that can result from concentrated localized delivery proximal to a tissue subject to such side effects, as is seen with repeated topical application of prostaglandin analog drops in glaucoma patients. The device's sustained delivery of drug can eliminate the use of topical eye drops, resulting in improved patient compliance, convenience, and subsequent efficacy. The drug can be incorporated when the device is manufactured, resulting in drug loaded depots with their openings on the anterior, lateral or any surface distal to the surface most proximal to the sclera or bulbar conjunctiva, of the topical ophthalmic drug delivery device.

In accordance with the present invention, a device for delivering active agent can be constructed such that the active agent is delivered to the body tissue (target tissue) over a prolonged period of time. More particularly, but not by way of limitation, the present invention pertains to biocompatible devices for localized delivery of active agents to the eye.

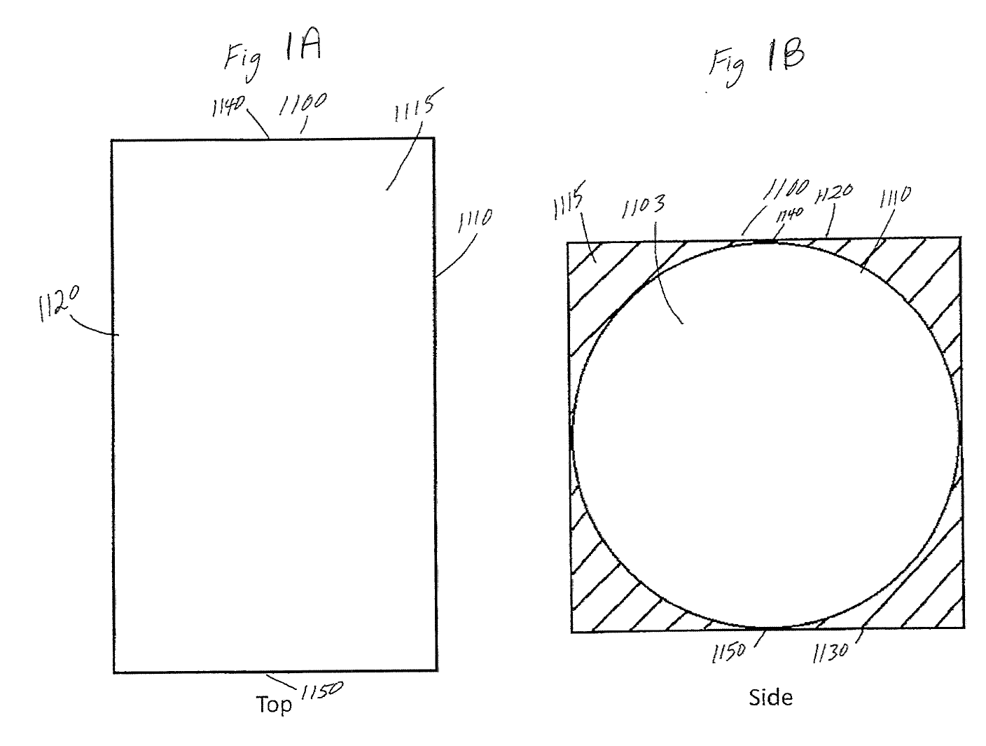

Now referring to FIGS. 1A and 1B, in one exemplary application, a device 1100 for delivering an active agent (e.g., a drug) is in the form of a topical ophthalmic drug delivery device 1100 according to one embodiment; however, it will be understood that the device 1100 is not limited to only being used in ophthalmic applications but instead can be used in other applications to treat other areas of the body. The drug delivery device 1100 is defined by a body 1110 that has prescribed dimensions that allow placement in the eye in this particular exemplary application. The body 1110 is defined by a first surface or face 1120 and an opposite second surface or face 1130. A thickness of the body 1110 is defined as a distance between the first surface 1120 and the second surface 1130. The body 1110 includes a peripheral edge 1140 which in the illustrated embodiment is shown as being a side wall. The body 1110 contains a drug depot 1103 containing an active agent. As described herein, the drug depot 1103 represents the source of the active agent and can come in any number of different physical forms.

The drug depot 1103 can consist of only medication or a material such as a matrix containing medication, a tablet containing medication or an enclosed liquid containing medication. The form of the drug depot 1103 can thus be the same as the forms of the active agent 103 described herein. In accordance with the present invention, one or more surfaces of the drug depot 1103 include at least one surface covered by an erodible surface of uniform or varying thickness 1115. At points 1140 and 1150 or along a narrow strip of the entire length, the thickness can taper to zero, so that drug release could begin immediately upon placement in situ.

It will also be appreciated that the shape of the illustrated body 1110 is merely exemplary and the body 1110 can have other shapes. The body 1110 is formed such that it has a degree of flexibility to allow placement of the body 1110 at the target location where the target tissue is located. The body 1110 can thus have material characteristics that allow the body 1110 to at least generally or substantially adopt the shape of the target tissue to which the body 1110 is applied. For example, when used in ophthalmic applications, the body 1110 can adapt to the shape of the eye as discussed in more detail below.

It will also be appreciated that either the first or second surface 1120, 1130 can be placed against target tissue, with the opposite surface thus facing away from the target tissue. In a topical ophthalmic application, either the first or second surface 1120, 1130 can be placed against the tissue of the eye as described in more detail below.

It will be understood that while the body 1110 has symmetry about a central axis, the device 1100 is not limited to having such a characteristic and instead, the body 1110 can have an asymmetric construction.

It will therefore be understood that the device 1100 as well as the other devices described herein and illustrated in the accompanying figures can be formed of materials that are disclosed in the previously incorporated applications and can have structure characteristics that are disclosed in the above applications.

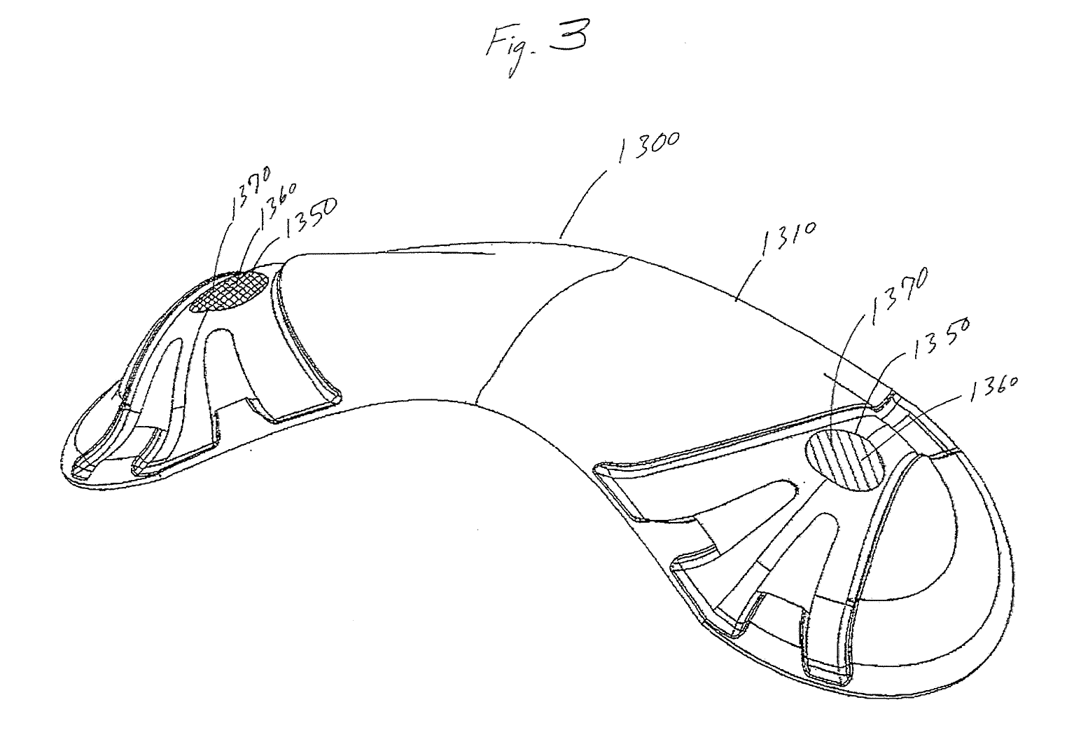

FIG. 3 shows a device 1300 according to a different embodiment in which the body 1310 contains an additional feature of a local recessed area (space) 1350, such as a pocket, well, reservoir, compartment, etc. One will appreciate that the device 1300 looks similar to the devices disclosed herein and the local recessed area 1350 can be the same as or similar to the local recessed area 250 as previously described herein.

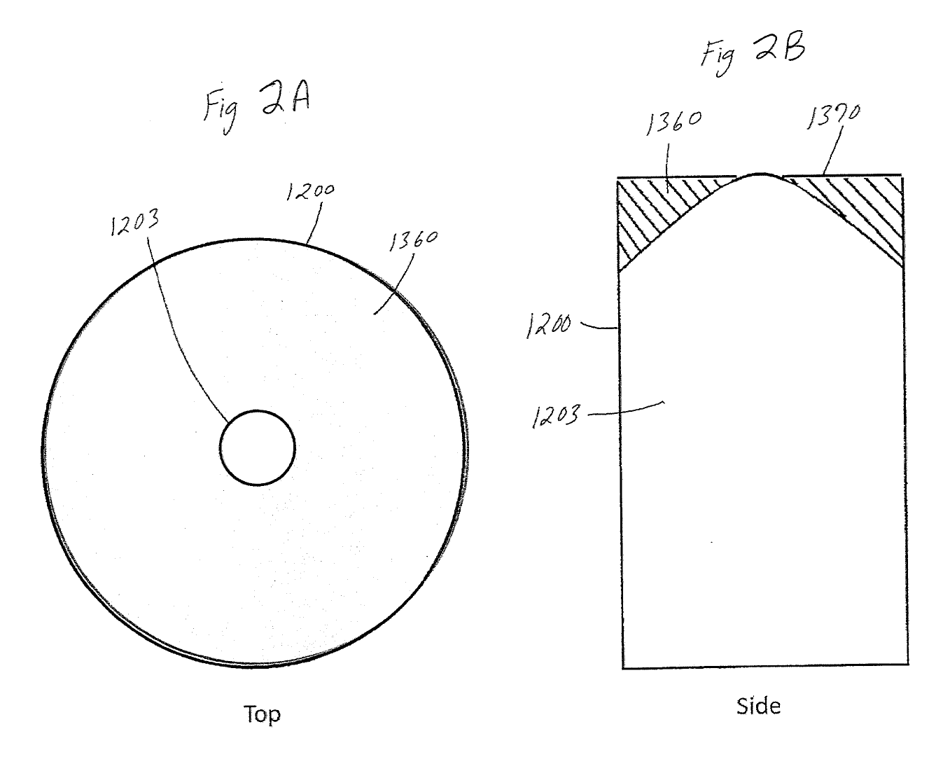

The local recessed area 1350 is located and formed such that it is in fluid communication with the surface. More specifically, the local recessed area 1350 receives and holds the active agent, or drug depot, which is identified in FIG. 2 as 1203. The active agent 1203 is placed in a drug depot 1203 positioned within the pocket, but not filling the local recessed area 1350. The depot can consist of only medication or a material such as a matrix containing medication, a tablet containing medication or an enclosed liquid containing medication. The geometry of the depot varies in volume as a function of pocket depth; that is the volume of the depot is least nearing the top, and greatest at the bottom of the local recessed area 1350. A biodegradable material is placed in the pocket “over” the depot to fill the pocket creating a composite structure 1360, said biodegradable material composite structure substantially impermeable to the medication in the depot. The modified drug depot will then either have a generally flat top 1370 with just a small portion of the drug depot 1203 comprising a portion of the exposed surface of the local recessed area 1350, or a generally flat top 1370 consisting entirely of the biodegradable material composite structure 1360 that is entirely over the drug depot 1203 below.

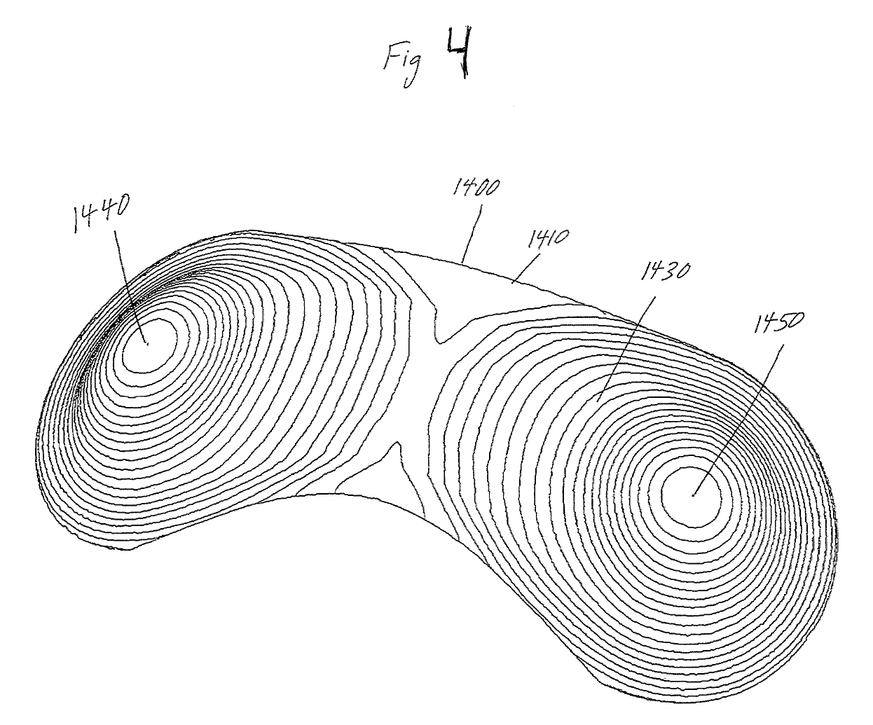

FIG. 4 shows the device 1400 in which the active agent is contained within the body 1410 (e.g., dispersed throughout a polymeric matrix that forms the body 1410) and is dispensed through any exposed portions of the surfaces thereof the surface being covered by an erodible surface of uniform or varying thickness 1430. At points 1440 and 1450, the thickness can taper to zero, so that drug release could begin immediately upon placement in situ.

It will be understood that in this aspect of the present invention, a controlling medication release from either a matrix type or drug depot type drug delivery device is disclosed. There are two operative approaches to the practice of the present invention; one applies to a device that is constructed entirely of a medication containing matrix, the second approach involves a device where the medication is localized within the device body.

In the case where the device body consists entirely of medication containing matrix the device construction can include two further elements, namely, (1) the device matrix body of a structure and geometry suitable for its intended placement location within the mammalian body; and (2) a conformal coating on the body with the coating consisting of an erodible material that varies in depth. The conformal coating will have one or more “holes” (openings/perforations) in the coating that exposes the underlying matrix. The number and size of the “holes” will vary depending on the geometry of the device, the matrix material, the chemical nature of the medication, the medication concentration and the release profile desired. Alternatively, the conformal coating may be non-uniform in thickness and the underlying matrix surface will be exposed as the thinnest portions of the coating degrade first.

The principle with this device construction is that the initial medication release will occur through the exposed matrix in the “holes” or as the thinnest layer of coating degrades to expose the underlying matrix surface. This will greatly reduce or eliminate the initial drug release “burst effect” that is commonly seen with conventional matrix devices. The conformal coating will slowly erode exposing more underlying matrix surface thus allowing more medication to be released roughly proportional to the amount of exposed matrix surface area. In this manner the medication release kinetics can be controlled in such a manner that release rate can be more constant over the service life of the device.

In addition to these two elements described for the matrix type device, an additional element can be useful in the construction of a depot type device;

an optional non-erodible barrier, substantially impermeable to the medication contained in the matrix, covering some portion of the device's surface.

In the case where the device body consists of the medication containing depot localized within the device body, the device construction can include at least four elements: (1) the device body of a structure and geometry suitable for its intended placement location within the mammalian body; (2) a pocket (local recessed area) in the device body proximate to the surface with said pocket having a portion open to the outside environment. The pocket can be in the form of a hole, cavity, well, chamber or recess of various simple or complex geometries, and may include features such as barbs, slots, grooves, threads, rings, tabs or nubs, to help contain and stabilize the drug depot contained therein; (3) a drug depot positioned within the pocket, but not filling the pocket. The depot can consist of only medication or a material such as a matrix containing medication, a tablet containing medication or an enclosed liquid containing medication. The geometry of the depot varies in volume as a function of pocket depth; that is the volume of the depot is least nearing the top of the pocket and greatest at the bottom of the pocket. If a cylindrical pocket is formed, two alternative simple geometries can illustrate examples of this definition and in particular, one can be a conical depot placed flat side down in the pocket; the other can be one half of a spherical depot placed flat side down in the pocket. The drug depot, in whatever shape, has a small portion that reaches the top or near the top of the pocket; and (4) a biodegradable material placed in the pocket “over” the depot to fill the pocket creating a biodegradable material composite structure that is substantially impermeable to the medication in the depot. The modified drug depot can either have a generally flat top with just a small portion of the drug depot comprising a portion of the surface area of the pocket top, or a generally flat top that is entirely over the drug depot below.

In addition to these four elements two additional elements can be useful in the construction of a depot type device: (1) an optional membrane or thin film placed over the pocket's opening to further regulate the release of medication from the pocket with the membrane totally or partially covering the pocket's opening; and (2) depending on the construction of the drug depot and the polymeric material utilized to construct the device body it may be necessary to render the walls and bottom of the pocket impermeable to diffusion of the medication. This will prevent unwanted diffusion of the medication into the device body and direct the release of medication through the top of the pocket and into the ocular environment.

The body of the device can be composed entirely of matrix that contains medication or the device may contain a localized medication depot. In either case, typically the body of the device is formed from a polymeric non-erodible material, preferably elastomeric in nature. In the case where the device body in its entirety is a medication containing matrix, the polymeric material is chosen primarily for its ability to provide the desired release kinetics. For a device with a localized drug depot, the body material itself may be chosen with more latitude. One important aspect of the body material in this case is its ability to resist diffusion of the medication into said body from the included pocket. Examples of polymeric materials useful in the practice of this invention are, but are not limited to, polyacrylates and methacrylates, polyvinyl ethers, polyolefins, polyamides, polyvinyl chloride, fluoropolymers, polyurethanes, polyvinyl esters, polysiloxanes and polystyrenes.

While typically the device's body is formed of non-erodible material, the body can also be constructed of biodegradable material more resistant to erosion than the matrix device's conformal coating or the biodegradable material in the drug depot pockets.

It will be appreciated that the depot 1103 can be any of the active agents disclosed herein and discussed with reference to active agent 103 in previous embodiments.

Erodible materials in the context of this invention are defined as organic materials that break down into simple chemicals commonly found in the body. More specifically, the term biodegradation is often used to describe polymers that break down when in contact with bodily fluids as defined below.

Biodegradation is the chemical breakdown of materials by exposure to a physiological environment. The materials may be organic, such as polymers, or inorganic, such as certain ceramics and silicas, and the degradation mechanism may be hydrolysis, enzymatic reaction or a combination of the two. In addition, the degradation process is also very sensitive to the pH of the environment. For the purposes of this invention the terms erodible, degradable, bioerodible and biodegradable all refer to the above defined process.

The following is a classification of biodegradable polymers that are suitable for use in the present invention: Synthetic Biodegradable Polymers including but not limited to Polyesters; Polyortho esters; Polyanhydrides; Polyamides; Polydioxanones; Polyoxalates; Polyacetals; Polyiminocarbonates; Polyurethanes; Polya-cyanoacrylates; Polyphosphazenes; and Natural Biodegradable Polymers including but not limited to Starch, Hyaluronic acid, Heparin, Gelatin, Albumin, Dextran and Chitisan.

Examples of biodegradable polymers commonly used in drug delivery that are suitable for use in the practice of the present invention include but are limited to: Polylactic acid, Polyglycolic acid, Lactic/glycolic acid copolymers, Polycaprolactone, Poly-hydroxybutyrate, Polyhydroxyvalerate, Polydioxanone, Polyiminocarbonate, Polyorthoesters, Polyanhydrides, Polyamides, Poly o-cyanoacrylates, maleic anhydride copolymers, Acrylamide-N,N′-methylenebisacrylamide, N-vinyl pyrrolidone-N,N′-methylenebisacrylamide, Fumaric acid/polyethylene glycol-N-vinyl pyrrolidone, Fumaric acidfdiglycolic acid-N-vinyl pyrrolidone, Fumaric acidlk.etomalonic acid-N-vinyl pyrrolidone, Fumaric acid/ketoglutaric acid-N-vinyl pyrrolidone, Poly(amino acids), Psuedopolyamino acids, Polyphosphazenes, Starch, Hyaluronic acid, Heparin, Gelatin, Albumin, Dextran and Chitisan.

Biodegradable polymers can be categorized into two groups on the basis of the mechanism or process by which they degrade. These processes are bulk degradation and surface degradation. In the case of polymers degrade in bulk. The rate of water penetration into the matrix is faster than the rate of polymer degradation. The process is a homogeneous one in which degradation occurs at a uniform rate throughout the polymer matrix. In contrast, for polymers which undergo surface degradation, the rate of water penetration into the matrix is slower than the rate of polymer degradation an example of such materials are the polyanhydrides. This process, therefore, is heterogeneous with degradation confined to a thin surface layer of polymer. For the purposes of the present application a preferred method of degradation is through continued erosion of the surface layers after installation of the device into the eye.

Once the device is constructed one option is to attach a membrane or thin film over the pocket to regulate the release of medication from the pocket. The membrane may totally or partially cover the pocket. This membrane or film can be chosen from among polymers that are permeable, to some degree, to the drug or medication in the drug depot. The membrane by definition restricts the flux of the drug or medication from the pocket. The membrane is utilized to tailor the release profile of the medication. Common membranes include ethylene vinyl acetate (EVA) polymers, silicones and poly(meth)acrylates. Other polymers could also be employed as useful membranes as well.

The intended drug diffusion path is from the exposed drug depot surface, directly to the ocular environment or through a thin release controlling membrane between the drug depot surface and the ocular environment. It is understood that unless it is prevented drug from the depot will diffuse out of all surfaces of the drug depot. This will lead to drug loss by diffusion into the main body of the device. The drug in the body of the device is then generally not available to provide therapeutic value to the patient. This non-productive drug diffusion must be eliminated or at least decreased by one order of magnitude to maximize the drug flux through the drug depot surface adjacent to the ocular environment. If the drug pocket is in the form of a cylinder then it would be necessary to place a barrier on the side surface and the flat bottom surface of the cylinder. This would then allow drug to diffuse from the top surface only. This top surface would then be placed adjacent to the device surface to direct drug flux out of the device and into the ocular environment.

One technique to provide directional flux of the drug is to cast the depot into a plastic container such as a barrel with an open top. There are many plastics that are excellent barriers such as polymethyl methacrylate, polyimide, Teflon® and polypropylene to name a few. The one drawback to this approach is the plastic container would be difficult to manufacture because of the small sizes required. Another drawback is that the physical size of any plastic container will increase the overall volume of the drug depot. This is not desirable given the small size of the ocular device itself. Another approach is to form the diffusion barrier around the drug depot by applying a very thin film of the barrier. It is possible to apply a thin silica coating over the drug depot by chemical means but this may be a costly process. The preferred method of creating a barrier on the drug depot is the application of Parylene, a well known barrier thin film. Parylene is the trade name for a variety of chemical vapor deposited poly (p-xylylene) polymers used as moisture and dielectric barriers. Among them, Parylene C is the most popular due to its combination of barrier properties, cost, and other processing advantages. Parylene is self-initiated (no initiator needed) and un-terminated (no termination group needed) with no solvent or catalyst required. Its polymerization occurs at a very low pressure and at near room temperature. The entire process is known as CVD, or Chemical Vapor Deposition. The resulting parylene film which has bonded during the deposition process becomes a thin, microns in thickness, protective coating. Parylene conforms to almost any exposed surface and unlike typical liquid coatings, it penetrates small crevices and uniformly coats surfaces such as sharp points, cavities, edges, corners and even minute pores. Additionally, Parylene provides barrier protection against organic as well as inorganic compounds.

The devices of this invention can be fabricated from polymer based materials. For a matrix device the drug or medicinal agent can either be in a dissolved and/or dispersed state within this polymeric matrix. In one embodiment the drug or medicinal agent is compounded into a preformed polymer where it may be in the dissolved or dispersed state. The device is then formed from this drug containing polymer. Examples of useful polymer matrices are ethylene vinyl acetate and acrylic based polymer materials. In another embodiment, the drug or medicinal agent can be compounded into a reactive system. That system may be a monomer or macromer where the drug or medicinal agent is in the dissolved or dispersed state. The liquid is then placed in a mold that bears the shape of the device. Polymerizing the system, typically through UV, visible light, heat or a combination of these means, then forms the device. Examples of useful reactive systems would include the use of liquid acrylic monomers or reactive silicone pre-polymers.

One preferred manufacturing process for producing the matrix drug delivery devices of this invention is cast molding. In this process a drug or medicinal agent is dissolved and/or dispersed in a monomer mixture and placed in a plastic casting mold bearing the geometry of the ocular device. Thermal exposure, UV exposure or a combination of both polymerizes the monomer. The device is then removed from the mold. Post processing may be required, for example, edge finishing. The biodegradable coating is then applied to the finished matrix device.

The devices of this invention can also be constructed with a pocket or opening in the device body proximate to the surface with said pocket having a portion open to the environment outside the device. The pocket will be partially filled with a medication depot with the pocket's opening partially or completely filled with a biodegradable polymer. Optionally, the pocket can also be covered with a medication release controlling membrane.

A single device can contain multiple pockets. Each pocket can be partially filled with a drug depot, each with a different depth of biodegradable polymer being used to fill the pocket's opening. So configured the device could provide sequential individual bursts of drug release rather than one large burst beginning upon the application of the device. Alternatively, the pockets could be covered with erodible material of differing eroding times and this could also provide sequential release characteristics. The device could also be configured to deliver multiple drugs. These configurations could also be combined such that the device releases one drug initially and a different drug at a later time. With multiple pockets, the device could also be configured to alternate or overlap the timing of the release of different drugs.

The devices of this invention can also be constructed with a drug depot that is entirely enclosed within the body of the device and that is enabled to transport drug from the depot to at least some portion of the body's surface.

In a further aspect, the present invention comprises an ophthalmic drug delivery device including a body with a surface for placement proximate a sclera and a pocket or cavity having an opening to the scleral surface. A drug depot comprising a pharmaceutically active agent is disposed in the pocket. Biodegradable (bioerodible) material can be disposed across and over the drug depot and the thickness of the biodegradable material can be non-uniform (thicker at edges of the pocket of the device) (FIGS. 18A and 18B). A window where the biodegradable material is absent can be provided where the drug depot is exposed.

In a further aspect, the present invention comprises a method of delivering a therapeutic agent to an eye having a sclera, a Tenon's capsule, and a posterior segment. A drug delivery device comprising a body having a therapeutically active agent disposed therein is provided. The device is disposed on an outer surface of the sclera, below the Tenon's capsule, and proximate the posterior segment.

The device body can be fabricated from a polymeric material by a molding process. This would include, but not be limited to, cast molding, standard injection molding, liquid injection molding, compression molding and transfer molding.

The matrix containing medication depot can be fabricated separately from the device body in a desired configuration and then placed in the pocket. Alternatively, the matrix containing medication depot can be formed in situ in the pocket. In either case, once the matrix containing medication depot is in the pocket the biodegradable polymer is introduced into the pocket to at least partially cover the exposed surface of the depot. At this point, if applicable, the release controlling membrane can be applied over the pocket's opening to the device's surface.

Another method for fabricating the device bodies, especially those with a pocket, is molding. This would include, but limited to, cast molding, standard injection molding, liquid injection molding, compression molding and transfer molding.

Post processing may be required, for example edge finishing. In the case of an ocular device, polypropylene casting molds are preferred. One preferred material is a polypropylene resin with a melt flow index above 20. One polypropylene resin is PP 1901-01 which has a melt flow index of about 34 g/10 min. With melt flows above 20 gm/10 min intricately shaped casting molds can be injection molded with excellent replication of part dimensions.

Another preferred manufacturing process for producing the drug delivery devices of this invention is liquid injection molding which is particularly well suited for siloxane materials. The siloxane prepolymer is mixed with a polymerization catalyst at room temperature then injected into a hot mold to cure. After the device is cured it is removed from the mold.

Post processing is sometimes required to remove flash and/or to contour the parting line. For the topical devices of this invention, the edge profile is critical in providing device comfort and fit. The edges of these devices can be shaped and contoured utilizing standard polishing techniques currently utilized in the ophthalmic industry. More preferred is the use of laser edging to form a smooth, well-contoured edge.

It will be appreciated that the active agent can be delivered over a prescribed period of time (prolonged period of time) that depending upon the particular agent and the application, the time period can range from a number of days (e.g., 1 week) to a number of months (e.g., 90 days or more, 120 days or more, etc.)

What is claimed is:

1. A device for delivering an active agent to target tissue over a prescribed period of time to target tissue, the device comprising:
a body;
a depot that contains active agent and is supported by the body, the depot including a first surface; and
an erodible member disposed at least along a portion of the first surface of the depot, the erodible member having a varying thickness across the first surface of the depot so as to control the release of the active agent from the depot over the prescribed period of time.

2. The device of claim 1, wherein the body is configured for placement in the eye and the erodible member is formed of a material that erodes over time in ocular fluid.

3. The device of claim 1, wherein the body includes a local recessed area that receives the depot with the erodible member being exposed along an exterior of the body.

4. The device of claim 1, wherein the first surface has a convex shape with the erodible member being at a minimum thickness at an apex of the depot.

5. The device of claim 4, wherein the erodible member has a greater thickness at peripheral edges of the depot.

6. The device of claim 1, wherein there are a plurality of depots and a plurality of associated erodible members, the erodible members having different cross-sectional profiles so as to provide different release profiles over time for the active agent.

7. The device of claim 6, wherein at least one erodible member is constructed to dispense the active agent immediately upon placement at a site that includes bodily fluids.

8. The device of claim 1, wherein the first surface is non-planar in nature and an inner surface of the erodible member is non-planar in nature and has an opposite profile relative to the first surface of the depot, while an outer surface of the erodible member is at least substantially planar.

9. The device of claim 1, wherein the first surface has a concave shape with the erodible member being at a maximum thickness at a center of the depot.

10. The device of claim 1, wherein the erodible member is formed of a material that prevents the active agent from passing therethrough prior to at least a portion of the erodible member eroding to a degree that an open conduit is formed through the erodible member to the exterior.

11. The device of claim 1, wherein the erodible member has a non-uniform cross-sectional profile that is selected in view of a desired release rate of the active agent relative to the prescribed period of time.

12. The device of claim 1, wherein the depot and erodible member are stable outside of the body of the device and represent an insert that can be disposed within a local recessed area formed in the body.

13. The device of claim 12, wherein an exterior surface of the body of the device includes a recessed open canal formed therein, the canal being open at least at one end and along a top thereof, the canal intersecting the local recessed area so as to guide bodily fluid into contact with the erodible member.

14. The device of claim 1, wherein the first surface of the depot faces the target tissue when the device is applied.

15. The device of claim 1, wherein the erodible member is formed along the entire first surface so as to completely cover the depot.

16. The device of claim 1, wherein the erodible member does not cover the entire first surface of the depot, thereby leaving an area of the depot to be exposed to the exterior.

17. An ocular device for delivering an active agent to scleral tissue over a prescribed period of time to target tissue, the device comprising:
a body;
a depot that contains active agent and is supported by the body, the depot including a first surface; and
an erodible member disposed at least along a portion of the first surface of the depot, the erodible member having a varying thickness across the first surface of the depot so as to control the release of the active agent from the depot over the prescribed period of time.

18. The device of claim 17, wherein the erodible member is disposed along additional surfaces of the depot besides the first surface.

19. The device of claim 17, wherein the erodible member is formed along the entire first surface so as to completely cover the depot.

20. The device of claim 17, wherein the erodible member does not cover the entire first surface of the depot, thereby leaving an area of the depot to be exposed to the exterior.

21. The device of claim 17, wherein the body includes a local recessed area that receives the depot with the erodible member being exposed along an exterior of the body.

22. The device of claim 17, wherein the first surface has a convex shape with the erodible member being at a minimum thickness at an apex of the depot.

23. The device of claim 17, wherein the erodible member is formed of a material that prevents the active agent from passing therethrough prior to at least a portion of the erodible member eroding to a degree that an open conduit is formed through the erodible member to the exterior.

24. The device of claim 17, wherein the erodible member has a non-uniform cross-sectional profile that is selected in view of a desired release rate of the active agent relative to the prescribed period of time.
